(19) United States
(12) Patent Application Publication
(10) Pub. No.: US 2012/0022321 A1
(43) Pub. Date: Jan. 26, 2012

(54) Coated Pelvic Implant Device and Method

(75) Inventors: Thomas Q. Dinh, Minnetonka, MN (US); Rosa K. Canifax, Bloomington, MN (US)

(73) Assignee: AMS RESEARCH CORPORATION, Minnetonka, MN (US)

(21) Appl. No.: 13/188,797

(22) Filed: Jul. 22, 2011

Related U.S. Application Data
(60) Provisional application No. 61/366,812, filed on Jul. 22, 2010.

(51) Int. Cl. A61F 2/00

(52) U.S. Cl. 600/37

(57) ABSTRACT

Implant systems and methods are provided to include a treatment material with a pelvic implant device. The pelvic implant device, such as an incontinence sling, can include the treatment coating combination of polycarbonate (PC) and an infection prevention material, such as InhibiZone® (IZ) technology. The treatment material can be coated onto, or impregnation or integrated with, polypropylene mesh in order to prevent infection and promote healing.

### PRIORITY

This Application claims priority to and the benefit of U.S. Provisional Patent Application No. 61/366,812, filed Jul. 22, 2010, which is incorporated herein by reference in its entirety.

### FIELD OF THE INVENTION

The invention relates generally to surgical devices and methods and, more particularly, to a pelvic implant device coated with a treatment material.

### BACKGROUND OF THE INVENTION

Pelvic health for men and women is a medical area of increasing importance, at least in part due to an aging population. Examples of common pelvic ailments include incontinence (fecal and urinary), pelvic tissue prolapse (e.g., female vaginal prolapse), and conditions of the pelvic floor.

Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

Urinary incontinence can be characterized by the loss or diminution in the ability to maintain the urethral sphincter closed as the bladder fills with urine. Male or female stress urinary incontinence (SUI) generally occurs when the patient is physically stressed. Physical stresses that can cause urinary incontinence include jumping, coughing, sneezing and laughing to name a few.

In its severest forms, vaginal vault prolapse can result in the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons. These procedures often involve lengthy surgical procedure times.

Many strategies have been implemented over the years to provide mesh implants adapted to enhance therapeutic support of the respective pelvic tissues. For instance, sling and other implant devices are known to provide support of the urethra or bladder neck in treating urinary incontinence in patients. Further, various mesh implants have been adapted to provide pelvic floor support to treat certain prolapse disorders.

While many of the above-identified methods and systems currently provide advantageous options for the treatment of SUI, or other ailments and disorders, desirable coated implant devices are needed.

### SUMMARY OF THE INVENTION

The invention relates generally to a mesh sling or pelvic implant device adapted for treating incontinence or other known pelvic disorders, e.g., male or female incontinence (fecal and urinary), pelvic tissue prolapse (e.g., female vaginal prolapse), and other conditions of the pelvic floor.

Various embodiments of the present invention can be employed with or by using existing or known incontinence sling materials, devices or procedures. In one embodiment, the pelvic implant device includes a treatment material that can combine polycarbonate (PC) and an infection prevention material or coating, such as InhibiZone® (IZ) technology. The treatment material can be coated onto, or impregnation or integrated with, polypropylene mesh in order to prevent infection and promote healing. IZ is a material combination of rifampin and minocycline antibiotic drugs or agents, and provides an advantageous antibiotic or antimicrobial surface treatment. The implant of the present invention can include the antimicrobials via advantageous coating and curing to improve effective and sustained drug release. Other antibiotic treatment or coating materials can be included with, or in lieu of, the IZ technology to provide the desired treatment parameters for the implant device, or to include drug or agent eluding features.

Various polyester, polyurethane, nylon or polypropylene materials can be provided with the IZ component, or combined with other compatible and like materials to facilitate the coating process or to promote the treatment effectiveness of the applied IZ material.

While there are numerous techniques that can be employed to provide the treatment material with the implant device, embodiments of the coating procedures can include the following two methods: (1) coating at least a portion of the implant with a solution including both Polycarbonate (PC) and IZ (with varied available combination ratios) components; or (2) pre-coating at least a portion of the implant first with a PC component and then loading the PC coating with an IZ solution or component.

### BRIEF DESCRIPTION OF THE DRAWINGS

FIGS. 1-3 are plan views of various pelvic implant devices including a portion having a treatment coating in accordance with embodiments of the present invention.

FIG. 4 is a plan view of a mesh implant portion including a treatment coating in accordance with embodiments of the present invention.

FIG. 5 is a graph including exemplary coating and solution data for mesh implants having a treatment coating in accordance with embodiments of the present invention.

### DETAILED DESCRIPTION OF PREFERRED EMBODIMENTS

The present invention is directed to sling or other implant devices 10 including a treatment material 20. The treatment material 20 can be a coating included with the implant device or otherwise integrated with the implant 10. The combination of the implant device 10 and the treatment material 12 can be included with implants such as mesh sling devices to treat disorders in males and females, such as incontinence (urinary or fecal) or stress urinary incontinence (SUI) in particular. Embodiments of the present invention can be utilized to treat vaginal prolapse or other pelvic floor disorders as well.

Various tools, device structures, implants, components, methods and techniques described and depicted in U.S. Pat. Nos. 7,686,760, 7,500,945, 7,407,480, 7,351,197, 7,347,812, 7,303,525, 7,070,556, 7,025,063, 6,911,003, 6,802,807, 6,702,827, 6,691,711, 6,652,450, 6,648,921, and 6,612,977, International Patent Publication Nos. WO 2011/072148, WO 2008/057261 and WO 2007/097994, and U.S. Patent Publication Nos. 2011/0124956, 2010/0261955, 2004/0039453, 2002/0151762 and 2002/0147382, are envisioned for use, in whole or in part, with the present invention. As such, the entire disclosures of the above-referenced patents and publications are incorporated herein by reference in their entirety.

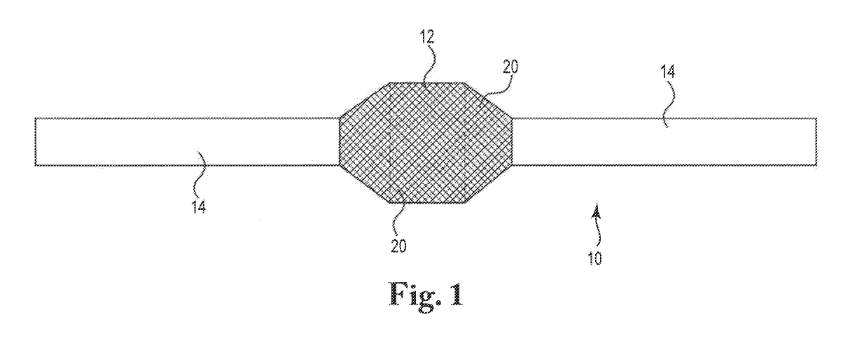

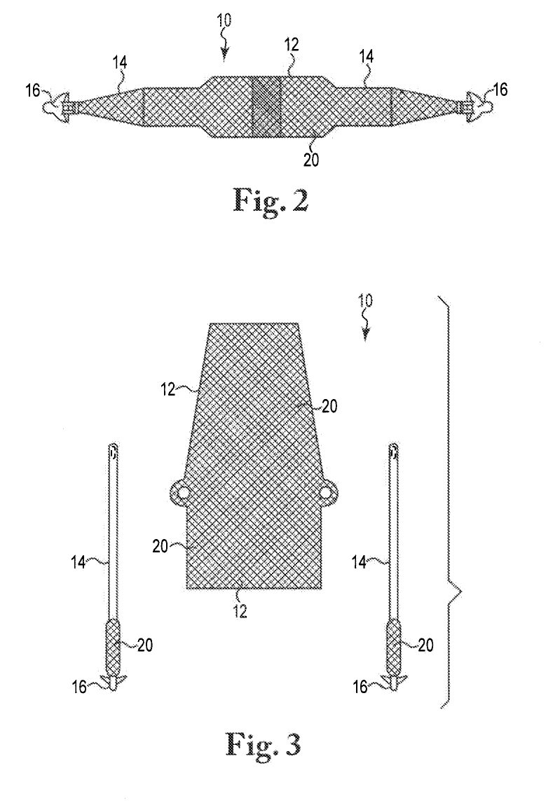

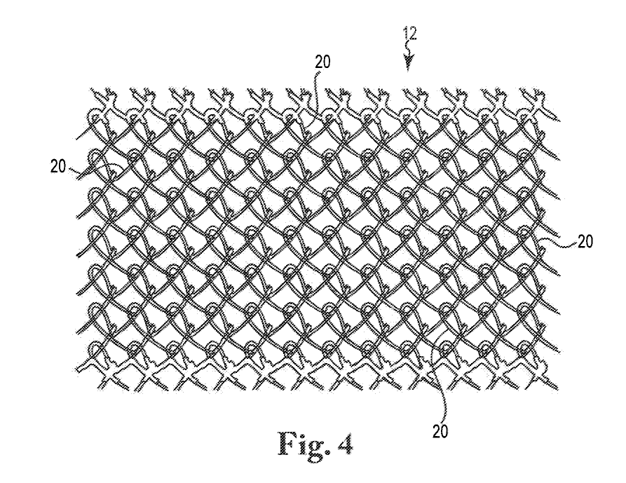

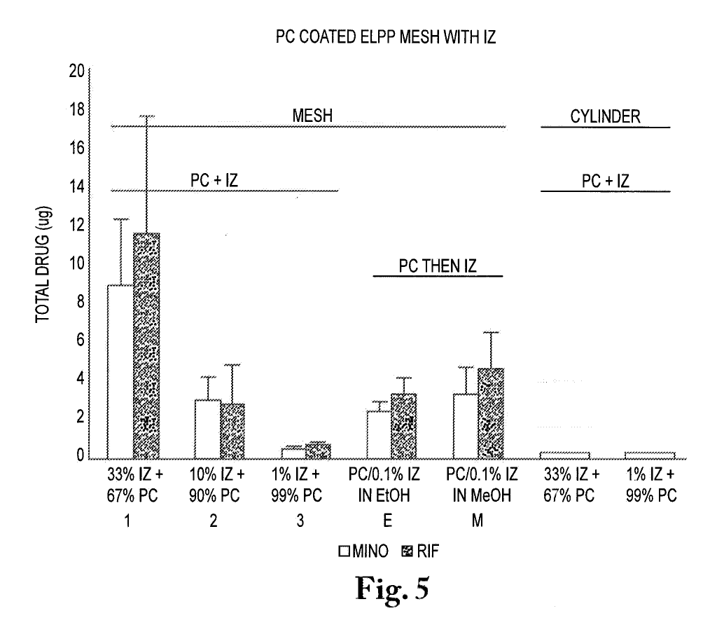

Referring generally to FIGS. 1-5, various embodiments of the sling or implant device 10 are shown. In general, the implants 10 can include a tissue support portion 12 and one or more extension or arm portions 14. In certain embodiments, ends of the extension portions 14 can provide anchoring or fixation out through external incisions in the patient (e.g., abdominal or perineal incisions). Other embodiments can include tissue anchors 16 at ends of the respective extension portions 14 to facilitate fixation to endopelvic tissue adjacent the target support area (e.g., urethra, bladder neck, rectum, etc.), as shown in FIGS. 2-3.

The one or more extension portions 14 can be included to span between or link the support portion 12 and the respective anchoring portions 16. Various portions of the implant 10 can be constructed of compatible polymer materials, e.g., woven, shaped, molded or otherwise formed into or from a generally planar film or sheet material. Portions of the sling 10, such as the support portion 12, can be formed of a mesh material (woven or non-woven), or formed or patterned by way of a polymer molding process to create a unitary generally homogeneous non-woven, or non-knitted, device or construct. Formation from a sheet or thin film can be achieved via laser cutting, die cutting, etching, punching, spraying, stamping and like procedures. Further, various embodiments of the implant 10 can be constructed of opaque or translucent polymer materials. The support portion 12 is generally adapted to support organs or tissue, such as that required to treat urinary or fecal incontinence, including the bladder neck, urethra or rectum.

A urethral sling device may be integral, monolithic, or a composite of different components or segments of different synthetic or non-synthetic (e.g., “biologic”) portions. Suitable synthetic materials for a sling include polymerics, metals, plastics and any combination of such materials. Examples of synthetic sling materials include polypropylene, cellulose, polyvinyl, silicone, polytetrafluoroethylene, polygalactin, Silastic, carbon-fiber, polyethylene, nylon, polyester (e.g., dacron) PLLA and PGA. The sling material may be resorbable, absorbable, or non-absorbable. Optionally, some portions may be absorbable and other portions may be non-absorbable. Further, mesh sling embodiments of the implant 10 can include a plurality of open pores (e.g., large pore polypropylene mesh (LLP) or extra large polypropylene mesh (ELLP)) to promote tissue in-growth and resist infection.

While there are numerous techniques that can be employed to provide the treatment material 20 with the implant device 10, embodiments of the present invention can include the following coating methods: (1) coating at least a portion of the implant with a solution including both polycarbonate (PC) and IZ (with varied available combination ratios) components; or (2) pre-coating at least a portion of the implant first with a PC component and then loading the PC coating with an IZ solution or component.

In one embodiment, the PC and IZ components are combinable for application to the device 10 substantially at the same time, in various available combination ratios, to a polypropylene mesh sling 10, or a sling constructed of like compatible material. Again, the coating 20 can be applied to all or select portions of the implant 10. In certain instances, it is beneficial to apply the coating 20 to the mesh support portion 12 of the sling 10, or other like portions that are regularly in contact with tissue or organs during deployment and implantation. The following Table 1 discloses IZ/PC substance combination ratios of: 33% IZ+67% PC; 10% IZ+90% PC; and 1% IZ+99% PC for exemplary material samples and average standard deviations (e.g., 1-4, 1-6 . . . 3-4).

TABLE 1

| | mino | rif | | mino | rif | | mino | rif |
|---|---|---|---|---|---|---|---|---|
| 33% IZ + 67% PC | | | 10% IZ + 90% PC | | | 1% IZ + 99% PC | | |
| 1-4 | 8.17 | 9.85 | 2-2 | 2.26 | 1.31 | 3-1 | 0.29 | 0.43 |
| 1-6 | 5.93 | 6.58 | 1-6 | 4.32 | 5.05 | 3-3 | 0.42 | 0.54 |
| 1-8 | 12.72 | 18.36 | 1-8 | 2.13 | 1.70 | 3-4 | 0.48 | 0.77 |
| avg | 8.9 | 11.6 | avg | 2.9 | 2.7 | avg | 0.4 | 0.6 |
| stdev | 3.5 | 6.1 | stdev | 1.2 | 2.1 | stdev | 0.1 | 0.2 |
| 33% IZ + 67% PC | | | | | | 1% IZ + 99% PC | | |
| 1-Cyl | 0.22 | 0.22 | | | | 3-Cyl | 0.22 | 0.22 |

Known solvents, such as Ethanol (EtOH) or Methanol (MeOH), can be used to provide the desired PC+IZ treatment solution combinations for applying to the target sling 10 polymer. Table 2 below illustrates exemplary elution and IZ+PC ratio sample data for various embodiments of the combination treatment coating 20. Again, the IZ component can include minocycline (mino) and rifampin (rif) to present the desired infection prevention effect.

TABLE 2
calibration curve (IZRC.M)
minocycline (ug/ml) = 3.54789 * area (mAU · s) − 5.95508
rifampin (ug/ml) = 3.78845 * area (mAU · s) − 8.86618
elution samples 1 mL PBS pH = 7.0. samples coupons evaporated PC + IZ
soluution in aluminum pan. samples cut to 1 cm2
Day 1

| 1% IZ + 99% PC | | | 10% IZ + 90% PC | | | 33% IZ + 67% PC | | |
|---|---|---|---|---|---|---|---|---|
| | mino | rif | | mino | rif | | mino | rif |
| elution 1-1 | 49.30 | 14.87 | elution 10-1 | 295.67 | 671.55 | elution 30-1 | 1047.23 | 8070.00 |
| elution 1-2 | 51.40 | 7.07 | elution 10-2 | 267.70 | 460.32 | elution 30-2 | 846.68 | 4073.78 |
| elution 1-3 | 18.63 | 13.29 | elution 10-3 | 655.91 | 1247.66 | elution 30-3 | 625.30 | 2894.06 |
| avg (area) | 39.8 | 11.7 | avg (area) | 406.4 | 793.2 | avg (area) | 839.7 | 5012.6 |
| stdev (area) | 18.3 | 4.1 | stdev (area) | 216.5 | 407.5 | stdev (area) | 211.0 | 2712.7 |
| avg | 292 | 1890 | avg | 138 | 292 | avg | 292 | 1772 |
| stdev | 69 | 1019 | stdev | 71 | 146 | stdev | 69 | 956 |

Upon creating the desired treatment solution of PC+IZ, the coating 20 can be applied to the target sling implant portion (e.g., support portion 12) for a desired period of time. For instance, various procedures will apply the coating 20 by dipping the select portion of the sling 10 in the coating 20 for a period of approximately 10-60 seconds. Upon reaching the desired coating application period, the implant 10, with applied coating 20, is generally placed in an oven or other system for a determined curing period (e.g., 2-24 hours) to complete the coating application process. Other time periods and application procedures known to those of ordinary skill in the art for coating polymer materials can be employed without deviating from the spirit and scope of the present invention.

In other embodiments, the PC coating can be first applied to the sling implant 10, cured for a determined period, then loaded with the IZ substance component to complete the treatment coating process. As shown in Table 3, exemplary minocycline (mino) and rifampin (rif) combination data is provided to demonstrate a total IZ loading of 0.1% (e.g., using EtOH and MeOH solvents) onto the existing PC coating. Of course, other coating combinations, ratios, solvents and antibiotic loading alternatives are envisioned for obtaining the treatment material coating 20 in accordance with embodiments of the present invention.

TABLE 3

| PC/0.1% IZ in EtOH | | | PC/0.1% IZ in MeOH | | |
|---|---|---|---|---|---|
| | mino | rif | | mino | rif |
| EtOH-4 | 1.94 | 2.60 | MeOH-1 | 4.74 | 6.73 |
| EtOH-5 | 2.23 | 3.01 | MeOH-2 | 2.12 | 3.13 |
| EtOH-6 | 2.88 | 4.18 | MeOH-5 | 2.90 | 3.84 |
| avg | 2.3 | 3.3 | avg | 3.3 | 4.6 |
| stdev | 0.5 | 0.8 | stdev | 1.3 | 1.9 |

The process of applying the PC coating prior to loading the antibiotic component onto a portion of the sling implant 10 can provide relatively higher loadings-per-amount, and can simplify the manufacturing process while simultaneously lower processing costs and achieving advantageous bioactivity for the final treatment coating 20.

While available and exemplary coating or impregnation percentages, agent/drug percentages, concentrations, bacterial growth results and material loading options available for use with the sling implants 10 of the present invention are detailed and examined herein, various other materials and procedures consistent with the objectives of the present invention are envisioned as well. Other antibiotics, antimicrobials, and synthetic or natural polymers, degradable or non-degradable, can be employed for coating or constructing portions of the sling implants 10 of the present invention. The resulting sling implants 10 will be constructed of a material or material combinations to deliver antibiotics or other drug treatments to the supported or targeted tissue, to prevent infection and promote healing.

The implant device 10 including the treatment material 20 itself can be applicable for use with any type of implant surgery that treats any particular condition, including but not limited to general and specific conditions relating to incontinence or pelvic prolapse conditions. Many implant products are presently available commercially that include mesh or sling portions ideal for incorporating the treatment material 20 of the present invention, including the following products manufactured and sold by American Medical Systems of Minnetonka, Minn.: the Perigee® products for the treatment of cystocele; the Apogee® products for treating enterocele, rectocele, and vaginal vault prolapse; the MiniArc®, MiniArc® Precise, Monarc® Sparc®, and BioArc® products for treating female incontinence; the Elevate® products for treating vaginal and vault prolapse; the InVance® and AdVance® products for treating male incontinence; and the UroLume® products for treating urinary obstructions.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. An implant device for treating a pelvic condition, comprising:
a support portion adapted to support pelvic tissue;
at least one extension portion; and
a treatment coating applied to at least the support portion, the treatment coating including a first polycarbonate component and a second antibiotic component.

2. The implant device of claim 1, wherein at least the support portion is constructed of a polypropylene mesh material.

3. The implant device of claim 2, wherein the polypropylene mesh material is a large pore polypropylene mesh material.

4. The implant device of claim 1, wherein the second antibiotic component includes a rifampicin antibiotic component and a minocycline antibiotic component.

5. The implant device of claim 1, further including one or more anchor devices provided at an end portion of the at least one extension portion.

6. The implant device of claim 1, wherein the at least one extension portion includes two or more extension portions.

7. The implant device of claim 1, wherein the treatment coating is applied to the support portion and the at least one extension portion.

7. The implant device of claim 1, wherein the first polycarbonate component is applied to at least the support portion prior to applying the second antibiotic component.

8. The implant device of claim 1, wherein the first polycarbonate component and the second antibiotic component are applied to at least the support portion in a single coating.

9. The implant device of claim 1, wherein the support portion is adapted to support tissue for treating urinary incontinence.

10. The implant device of claim 1, wherein the support portion is adapted to support tissue for treating fecal incontinence.

11. The implant device of claim 1, wherein the support portion is adapted to support tissue for treating vaginal prolapse.

12. The implant device of claim 1, wherein at least the support portion is constructed of a plurality of woven filament members to define a plurality of open pores.

13. The implant device of claim 1, wherein at least the support portion is formed from a generally planar unitary film material to include a plurality of open pores.

14. A urinary incontinence sling implant, comprising:
a polypropylene mesh support portion adapted to support pelvic tissue;
at least one extension portion;
a first polycarbonate coating applied to at least a portion of the mesh support portion; and
a second antibiotic coating applied over the first polycarbonate coating, the second antibiotic coating including a rifampicin antibiotic component and a minocycline antibiotic component.

15. The sling implant of claim 14, further including one or more anchor devices provided at an end portion of the at least one extension portion.

16. The sling implant of claim 14, wherein the at least one extension portion includes two or more extension portions.

17. The sling implant of claim 14, wherein the at least one extension portion is constructed of a polypropylene mesh material.

18. The sling implant of claim 14, wherein the first polycarbonate coating is applied to the support portion and the at least one extension portion.

19. The sling implant of claim 14, wherein at least the support portion is constructed of a plurality of woven filament members to define a plurality of open pores.

20. The sling implant of claim 14, wherein at least the support portion is formed from a generally planar unitary film material to include a plurality of open pores.
